(19) United States
(12) Patent Application Publication
(10) Pub. No.: US 2019/0083622 A1
(43) Pub. Date: Mar. 21, 2019

(54) CANNABINOID FORMULATIONS AND METHODS INCLUDING THE ANTIOXIDANT C60

(71) Applicants: Joshua Raderman, Boulder, CO (US); Kevin H. Fortin, Williams, IN (US)

(72) Inventors: Joshua Raderman, Boulder, CO (US); Kevin H. Fortin, Williams, IN (US)

(21) Appl. No.: 16/160,715

(22) Filed: Oct. 15, 2018

Related U.S. Application Data
(60) Provisional application No. 62/572,748, filed on Oct. 16, 2017.

(51) Int. Cl. A61K 47/06; A61K 31/047; A61K 31/192; A61K 31/352; A61K 47/44; A61K 9/107

(52) U.S. Cl. CPC A61K 47/06; A61K 31/047; A61K 9/107; A61K 31/352; A61K 47/44; A61K 31/192

(57) ABSTRACT

A cannabinoid formulation including a carbon-based antioxidant. Preferably, the formulation is a colloid including C60 and cannabinoids in a lipid-based excipient. The formulation improves shelf life of cannabinoids and inhibits degradation of the cannabinoids over time due to oxidation. The present invention further sequesters, neutralizes, or inhibits, oxidative free radicals in vivo. The action of the C60 prepares the body on a cellular level to enhance the bioactivity of the cannabinoids. The function of the C60 in combination with cannabinoids greatly reduces inflammation, improves metabolism, inhibits free radical degradation of telomeres, and has many other health benefits. The C60 in combination with the cannabinoids enhances the function of the cannabinoids by improving latency and efficacy in vivo.

### FIELD OF THE INVENTION

The present invention encompasses cannabinoid formulations including the antioxidant carbon 60 (C60 or C₆₀), more particularly the invention includes using carbon 60 to improve efficacy of cannabinoid formulations.

### BACKGROUND AND SUMMARY OF THE INVENTION

Carbon 60 is a molecule formed in a cage like structure that retains the shape of a soccer ball. The shape is particularly described as a buckminsterfullerne, or bucky ball. It has the chemical formulation C₆₀. Having a slight electric charge on the surface is the primary mechanism for interacting with other molecules, including hydrogen (H). The molecule is generally stable, but can be hydrogenated, halogenated or oxygenated under certain conditions.

Carbon 60 exhibits a small degree of aromatic character and can readily undergo addition with hydrogen to give polyhydrofullerenes.

In 2012 a toxicity study by Tarek Baati and Fathi Moussa from the University of Paris, showed that C60 dissolved in olive oil was not toxic to rodents. In a video interview with Professor Fathi Moussa regarding the study, further information was provided regarding the toxicity study, and the method of action whereby the lifespan of the rodents was increased by 90% relative to controls when the animals were dosed with C60 olive oil. Many have concluded that C60 has utility in extending lifespan in mammals including humans. In addition to extending lifespan, reduction in the expression of various chronic diseases has also been documented due to the oral or topical administration of C60 in a lipid carrier.

One of the numerous benefits of the utilization of cannabinoids is to improve health and longevity. Many users of cannabinoids desire balance, optimal health, wellness and longevity. One problem with cannabinoids is that over time cannabinoids such as Tetrahydrocannabinol (THC), Cannabidiol (CBD) and any of the hundreds of other cannabinoids tend oxidize. This can be a problem when being processed, packaged, stored and shipped. Cannabinoids are defined broadly any bioactive substance that influences cannabinoid receptors (e.g. CB1, CB2 and others) in mammals.

It is desired is to improve bioactivity of cannabinoids at the cellular level, and to improve the amount of time a single oral dose of cannabinoids remains bioactive in vivo.

It is also desired is a way to improve stability and shelf life of cannabinoids.

It is further desired is to create a formulation that improves health and longevity in humans.

It is further desired is a way to reduce inflammation, the expression of chronic diseases, to improve health, reduce pain, and extend life in mammals including humans.

The present invention includes an antioxidant to the formulation to improve bioactivity of cannabinoids, and to improve shelf life of packaged cannabinoid formulations. The addition of C60 to the formulation inhibits degradation of the cannabinoids over time due to oxidation, particularly the acid forms of various cannabinoids including Tetrahydrocannabinolic acid (THCA), Cannabigerolic acid (CBGA), and Cannabidiolic acid (CBDA). In one embodiment the C60 is in an pure non-hydrogenated form, in another embodiment an aliquot of the C60 is hydrogenated so that a portion of the C60 are polyhydrofullerenes. It can be appreciated that processing the C60 into the formulation may cause some degree of hydrogenation, but does not structurally change the underlying carbon buckminsterfullerene structure. In a preferred embodiment, a negligible amount of the C60 takes the form of a polyhydrofullerene. In another embodiment, more than half of the C60 is hydrogenated to optimize anti-oxidative bio-activity.

The present invention sequesters, neutralizes, or inhibits, oxidative free radicals in vivo and in shelf stable formulations.

A formulation in accordance with the present invention improves health and longevity due to the reduction in telomere degradation, and also improves health and longevity by reducing the degradation of active cannabinoids in vivo, which improves bioactivity of these molecules.

The action of the C60 prepares the body on a cellular level to enhance the bioactivity of the cannabinoids. The function of the C60 in combination with cannabinoids greatly reduces inflammation, improves metabolism, inhibits free radical degradation of telomeres, and has many other health benefits. The C60 in combination with the cannabinoids enhances the function of the cannabinoids by improving latency and efficacy in vivo.

The present invention preferably includes C60 in a lipid-based cannabinoid formulation. This formulation can be delivered orally, via pulmonary delivery, sublingually via the oral mucosal membrane, or other mucosal membrane in the body. Delivery can be accomplished with a dry inhaler, a nasal spray, eye drops, ear drops, a tincture deliverable to the upper digestive tract, a vaporizer, or other method. Administration can be accomplished transdermal with a transdermal patch, or topical cream to address local skin conditions, joint pain, or other condition.

C60 is not typically soluble in lipids or aqueous solutions. Current delivery of C60 utilizes mechanical mixing that creates a suspension in oil such as coconut oil or olive oil. Mechanical mixing, however, may not yield a homogeneous mixture with a suitable shelf stability.

Many cannabinoids are lipophilic and hydrophobic to varying degrees. Accordingly, in one embodiment of the invention, the formulation is a colloid with a lipid-based excipient, cannabinoids, C60, and combinations thereof. In another embodiment, the formulation excludes cannabinoids, such as for regulatory reasons, but yields a C60 colloid in a lipid excipient achieved through method or process described herein.

The term “colloid” includes homogeneous, noncrystalline substance consisting of molecules or ultramicroscopic particles of one substance dispersed through a second substance. Colloids include gels, sols, and emulsions. In a colloid, the molecules, substances, or particles generally do not settle under normal circumstances and cannot be easily separated out by ordinary filtering or centrifuging like those in a suspension.

In one embodiment of the invention, the mixture is processed into a colloid having a homogeneous, noncrystalline, mixture consisting of bioactive amounts of C60, Cannabidiol (CBD), Tetrahydrocannabinol (THC), Cannabinol (CBN), other cannabinoids, terpenes and combinations thereof.

The excipient preferably includes a combination of small chain lipids, medium chain lipids, and large chain lipids. Preferably the excipient is a natural oil such as cold pressed coconut oil.

The formulation of the present invention may be manifested as a product such as a gel, sol, or emulsion. Delivery can be oral, pulmonary, trans-dermal, rectal, or through any other means conventional in the art.

The process of making the formulation into a colloid inhibits the formulated molecules, compounds and particles from settling in a way that the molecules are not readily separated out by ordinary filtering or centrifuging like those in a suspension. The process of making the formulation also stabilizes the cannabinoids from oxidation.

It can be appreciated that the coconut oil, or a specific lipid, can have measurable bioactivity and synergy with the bioactive molecules described herein. Accordingly, the term “excipient” includes carriers that are non-bioactive, and those with synergistic bioactive capabilities. Preferably, the excipient is a natural plant-derived oil. In various alternate embodiments, it can be appreciated that the excipient may be an animal derived lipid, combinations of numerous lipids, synthetic lipid, an engineered lipid, or combinations thereof.

In another embodiment, the excipient is an aqueous mixture, or purified water.

A product in accordance with the present invention may include a topical product, capsule, pill, tincture, vaporizable (smokable) oil, oral spray or nasal spray. It can be appreciated that other delivery methods can be devised in accordance with the present invention.

The processes and methods of the present invention are capable of creating a colloid or homogeneous mixture of C60 molecules and cannabinoids. Preferably this is accomplished in a lipid based cannabinoid formulation. In an alternate embodiment the processes and methods are applied to create a product having an aqueous cannabinoid formulation.

### Processing Using Ultrasound Generated from a Submerged Ultrasonic Transducer

A method of manufacturing the present invention includes utilizing low power ultrasonic energy directed at the mixture of extracted cannabinoid oil (or isolate in an excipient such as a carrier oil) and the C60. This excites the oil on a molecular level, In one embodiment an ultrasonic generator with 40 w of power at 60 Hz frequency excites the mixture. Next the frequency is altered to alter the rate of absorption of energy and to improve solubility or suspended homogeneity of the C60 and cannabinoids.

In one embodiment the acoustic frequency delivered by a submerged ultrasonic transducer at 1000 w of power and at a frequency of between zero and 7000 Hz yields improved homogeneity of the mixture, and improves solubility of the C60.

In another embodiment of the present invention the ultrasonic energy is delivered by a submerged piezoelectric device. The piezoelectric device is submerged into the mixture and an electric current is delivered to the piezoelectric device to achieve a desired resonance of the piezoelectric device. This delivers precisely regulated acoustic waves within the mixture to achieve improved solubility of the C60 within one minute to 8 hours under optimal conditions.

In another embodiment, a Clark Synthesis AQ339 Diluvio Underwater Speaker is submerged into a container including the mixture to deliver the acoustic energy. In another embodiment, the mixture is flowed past one or more of the speakers (or other ultrasonic devices) through a pipe to achieve continuous production of product.

### Processing Using Pressure Cycling

Another method includes utilizing Pressure Cycling Technology (PCT) and relying on cycles of hydrostatic pressure between ambient and ultra-high levels, up to 35000 psi and greater. Preferably, the cycled pressure is between ambient pressure and 5000 psi.

In one embodiment, the pressure is adjusted to a range that will not significantly degrade the lipids, C60 molecules or cannabinoids. The pressure can be adjusted to achieve desired effects for delivery of the cannabinoids. The pressure cycling (PCT) controls the molecular interactions between the C60, the cannabinoids, and the lipids. Hardware including an ultrasonic sensors system or a spectroscopy based sensor system can be used to measure and optimize the efficacy of pressure cycling during production. A feedback loop circuit can be employed to automate this process.

### Processing Using a High Shear Mixer

In one embodiment, the mixture of cannabinoids, lipids and C60 are processed utilizing a high-shear mixer yielding a suspension of cannabinoids, lipids and C60. In another embodiment of the invention mechanical actuation suspends the cannabinoids and C60 in the lipid. Mechanical actuation includes stirring or injecting sterile air homogenizes the mixture and suspends the cannabinoids and C60 in the lipids.

### Packaged Products

In one embodiment, the cannabinoids and C60 are processed in an aqueous solution and formed into a beverage. In another embodiment, a lipid-based colloid including cannabinoids and C60 is packaged as a pill, capsule, spray, tincture, or edible packaged product.

In another embodiment the present invention is a pharmaceutical product which can be taken orally, or injected. In one embodiment, the pharmaceutical product includes a single isolated cannabinoid and C60.

Each dose of a packaged product in accordance with the present invention includes a safe and bioactive amount of cannabinoids (e.g. between 1-100 mg) and a safe and bioactive amount of C60 of between 1-100 mg of C60. Various exemplary formulations are described below.

Coconut oil is a preferred excipient for numerous reasons including the ease of production, cost, synergistic bioactivity with cannabinoids including anti-inflammatory properties, and because of the its electrical conductivity of coconut oil due to the existence of free lipids. The electrical properties of coconut oil do not significantly interfere with the action of the C60 in scavenging free radicals.

### Systematic Testing of Colloid Arrangement During Processing and Feedback Loop

The present invention utilizes analytical techniques in the processing of the mixture of the present invention. During processing a control system having sensors using either ultrasonic device, or Nuclear Magnetic Resonance to determine the arrangement and distribution of the C60 in the mixture, as well as selected cannabinoids. In a suspension, a uniform or homogenous distribution is desired. The results of testing during processing using either technique can be fed back via a feedback loop with appropriate logic and control hardware and software to optimize the frequency and power of the ultrasonic transducer, or a pressure regulator and a pressure pump assembly, or a mixer, in any of the various embodiments of the invention.

### C60 and Cannabinoids

The structure of a buckminsterfullerene is a truncated icosahedron with 60 vertices and 32 faces (20 hexagons and 12 pentagons where no pentagons share a vertex) with a carbon atom at the vertices of each polygon and a bond along each polygon edge. The van der Waals diameter of a C60 molecule is about 1.01 nanometers (nm). The nucleus to nucleus diameter of a C60 molecule is about 0.71 nm. The C60 molecule has two bond lengths. The 6:6 ring bonds (between two hexagons) can be considered “double bonds” and are shorter than the 6:5 bonds (between a hexagon and a pentagon). Its average bond length is 0.14 nm. Each carbon atom in the structure is bonded covalently with 3 others.

C60 has notable bioactivity. C60 has a slight positive +2 charge that attracts negatively charged oxidative free-radicals and neutralizes them. Each C60 molecule absorbed through the skin or internally functions as an anti-oxidant, reducing numerous free-radicals rapidly. This occurs without the C60 molecule being changed or losing reductive potency. C60 rapidly resets and keeps on working. When the cells of the body are relieved of an existing free-radical oxidative burden, they can once again function at natural peak efficiency, increasing energy, performance and virility of the subject. It is expected that lifespan and functional longevity can be enhanced through internal use of C60.

Scientific studies on animals found C60 doubled some rodent life spans. Learning speed and memory were increased significantly. Age related cognitive decline and tumors were prevented. Potential negative effects of environmental toxins and radiation were minimized. Test animals administered C60 lived longer, vigorous and healthy lives. C60 does not exhibit toxicity at even high doses.

Telomere length is directly related to lifespan. Telomeres wrap the ends of the chromosomes and keep them stable. Oxidative stress has been found through scientific research to be a significant cause of telomere shortening, the main cause of aging and many degenerative and chronic diseases. C60 reduces oxidative radicals, which slows the shortening of telomeres and the aging process. In some studies telomeres have been elongated.

The structure of a buckminsterfullerene is a truncated icosahedron with 60 vertices and 32 faces (20 hexagons and 12 pentagons where no pentagons share a vertex) with a carbon atom at the vertices of each polygon and a bond along each polygon edge. The van der Waals diameter of a C60 molecule is about 1.01 nanometers (nm). The nucleus to nucleus diameter of a C60 molecule is about 0.71 nm. The C60 molecule has two bond lengths. The 6:6 ring bonds (between two hexagons) can be considered “double bonds” and are shorter than the 6:5 bonds (between a hexagon and a pentagon). Its average bond length is 0.14 nm. Each carbon atom in the structure is bonded covalently with 3 others.

The C60 molecule is extremely stable, and withstanding high temperatures and high pressures. The exposed surface of the structure can selectively react with other species while maintaining the spherical geometry. Atoms and small molecules can be trapped within the molecule without reacting.

### Cannabinoids

A cannabinoid is one of a class of diverse chemical compounds that acts on cannabinoid receptors in cells that alter neurotransmitter release in the brain. Ligands for these receptor proteins include the endocannabinoids, phytocannabinoids, and synthetic cannabinoids. The most notable cannabinoid is the phytocannabinoid tetrahydrocannabinol (THC), the primary psychoactive compound in Marijuana (Cannabis Sativa L). Cannabidiol (CBD) is another major constituent of the plant, particularly in Hemp (Cannabis Sativa L). There are at least 113 different cannabinoids isolated from cannabis, exhibiting varied effects.

Synthetic cannabinoids encompass a variety of distinct chemical classes: the classical cannabinoids structurally related to THC, the nonclassical cannabinoids (cannabimimetics) including the aminoalkylindoles, 1,5-diarylpyrazoles, quinolines, and arylsulfonamides as well as eicosanoids related to endocannabinoids.

### Cannabinoid Receptors

Cannabinoid receptors are common in animals, and have been found in mammals, birds, fish, and reptiles. At present, there are two primary types of cannabinoid receptors, termed CB₁ and CB₂. The human brain has more cannabinoid receptors than any other G protein-coupled receptor (GPCR) type.

CB₁ receptors are found primarily in the brain. CB₁ is also found in the human anterior eye and retina.

CB₂ receptors are predominantly found in the immune system, or immune-derived cells, with the greatest density in the spleen. CB₂ receptors appear to be responsible for the anti-inflammatory and possibly other therapeutic effects of cannabis seen in animal models.

Classical cannabinoids are derived from their respective 2-carboxylic acids (2-COOH) by decarboxylation (catalyzed by heat, light, or alkaline conditions). Oxidative radicals may transform one cannabinoid into another, or into metabolic byproducts. The present invention includes all naturally derived and synthetic cannabinoid combinations, including cannabinoids form Cannabis Sativa L. These include, but are not limited to: THC (Tetrahydrocannabinol), THCA (Tetrahydrocannabinolic acid), CBD (Cannabidiol), CBDA (Cannabidiolic Acid), CBN (Cannabinol), CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), CBE (Cannabielsoin), and CBT (Cannabicitran).

The structure of C60 is a buckminsterfullerene. C60 is a truncated icosahedron with 60 vertices and 32 faces (20 hexagons and 12 pentagons where no pentagons share a vertex) with a carbon atom at the vertices of each polygon and a bond along each polygon edge. The van der Waals diameter of a C60 molecule is about 1.01 nanometers (nm). The nucleus to nucleus diameter of a C60 molecule is about 0.71 nm. The C60 molecule has two bond lengths. The 6:6 ring bonds (between two hexagons) can be considered “double bonds” and are shorter than the 6:5 bonds (between a hexagon and a pentagon). Its average bond length is 0.14 nm. Each carbon atom in the structure is bonded covalently with 3 others.

The C60 molecule is stable, withstanding high temperatures and high pressures. The exposed surface of the structure can selectively react with other species while maintaining the spherical geometry. Atoms and small molecules can be trapped within the molecule without reacting. Selected carbon bonds of the C60 molecule can be replaced with carbon from other molecules.

Each of the cannabinoid compounds above may be in different forms depending on the position of the double bond in the alicyclic carbon ring. There is potential for confusion because there are different numbering systems used to describe the position of this double bond. Under the dibenzopyran numbering system widely used today, the major form of THC is called Δ⁹-THC, while the minor form is called Δ⁸-THC. Under the alternate terpene numbering system, these same compounds are called Δ¹-THC and Δ⁶-THC, respectively.

This bond (6:6) or (6:5) of the carbon 60 can be modified by replacing these carbon bonds with carbon from the alicyclic carbon ring, or a portion of the tail of a carbon chain of a cannabinoid, including the last carbon of the tail (the carbon most distal from the alicyclic carbon ring). This is accomplished through cycloaddition or cyclopropanation to create a functionalized C60 molecule. Thus utilizing and addition reaction to achieve a new C60 molecule is contemplated in accordance with the present invention. Using a Diels-Alder reaction or a Bingel reaction are also contemplated with the present combination of C60 and select classical cannabinoids. The use of a Bingel reaction improves solubility and electrochemical behavior of the resulting molecule.

In one embodiment a C60 6:6 or 6:5 bond is re-configured by bonding a carbon from the tail of a classical cannabinoid molecule such as THC. This addition reaction is characterized as: C₂₁H₃₀O₂+C60--->C₈₁H₃₀O₂.

In an alternate embodiment, the cyclopropanation of C60 and THC yields a molecule having the formula: C₈₀H₃₀O₂.

Most classical cannabinoids are 21-carbon compounds. However, some do not follow this rule, primarily because of variation in the length of the side-chain attached to the aromatic ring. In THC, CBD, and CBN, this side-chain is a pentyl (5-carbon) chain. In the most common homologue, the pentyl chain is replaced with a propyl (3-carbon) chain. Cannabinoids with the propyl side chain are named using the suffix varin, and are designated, for example, THCV, CBDV, or CBNV.

### Cannabinoids in Other Plants

Phytocannabinoids are known to occur in several plant species besides cannabis. These include Echinacea purpurea, Echinacea angustifolia, Acmella oleracea, Helichrysum umbraculigerum, and Radula marginata. The best-known cannabinoids that are not derived from Cannabis are the lipophilic alkamides (alkylamides) from Echinacea species, most notably the cis/trans isomers dodeca-2E,4E,8Z,10E/Z-tetraenoic-acid-isobutylamide. At least 25 different alkylamides have been identified, and some of them have shown affinities to the CB₂-receptor. In some Echinacea species, cannabinoids are found throughout the plant structure, but are most concentrated in the roots and flowers. Yangonin found in the Kava plant has significant affinity to the CB1 receptor. Tea (Camellia sinensis) catechins have an affinity for human cannabinoid receptors. A widespread dietary terpene, beta-caryophyllene, a component from the essential oil of cannabis and other medicinal plants, has also been identified as a selective agonist of peripheral CB₂-receptors, in vivo. Black truffles contain anandamide.

Most of the phytocannabinoids are nearly insoluble in water but are soluble in lipids, alcohols, and other non-polar organic solvents.

Cannabis plants can exhibit wide variation in the quantity and type of cannabinoids they produce. The mixture of cannabinoids produced by a plant is known as the plant's cannabinoid profile. Selective breeding has been used to control the genetics of plants and modify the cannabinoid profile. For example, strains that are used as fiber (commonly called hemp) are bred such that they are low in psychoactive chemicals like THC. Strains used in medicine are often bred for high CBD content, and strains used for recreational purposes are usually bred for high THC content or for a specific chemical balance.

Quantitative analysis of a plant's cannabinoid profile is often determined by gas chromatography (GC), or more reliably by gas chromatography combined with mass spectrometry (GC/MS). Liquid chromatography (LC) techniques are also possible and, unlike GC methods, can differentiate between the acid and neutral forms of the cannabinoids. There have been systematic attempts to monitor the cannabinoid profile of cannabis over time, but their accuracy is impeded by the illegal status of the plant in many countries.

Cannabinoids can be administered by smoking, vaporizing, oral ingestion, transdermal patch, intravenous injection, sublingual absorption, or rectal suppository. Once in the body, most cannabinoids are metabolized in the liver, especially by cytochrome P450 mixed-function oxidases, mainly CYP 2C9. Thus supplementing with CYP 2C9 inhibitors leads to extended intoxication.

Some is also stored in fat in addition to being metabolized in the liver. Δ⁹-THC is metabolized to 11-hydroxy-Δ⁹-THC, which is then metabolized to 9-carboxy-THC.^{[40]} Some cannabis metabolites can be detected in the body several weeks after administration. These metabolites are the chemicals recognized by common antibody-based “drug tests”; in the case of THC or others, these loads do not represent intoxication (compare to ethanol breath tests that measure instantaneous blood alcohol levels), but an integration of past consumption over an approximately month-long window. This is because they are fat-soluble, lipophilic molecules that accumulate in fatty tissues.

In one embodiment, the formulation is used in conjunction with photonic therapy. In the late 1890's Niels Ryberg Finsen won a Nobel Prize for his use of photonic Therapy to treat smallpox and lupus. Photonic therapy is proven to heal injuries up to 60% faster than traditional approaches and has been used by astronauts. The C60 molecule has the capability of absorbing green spectrum light, transforming it, and re-releasing both blue and red light in solution. This formulation may be used in vivo to deliver photonic light in the red spectrum (630-800 nm) deep within the body. This may have the benefit of increased collagen production in vivo to reduce arthritic discomfort and improve dermal health, as well as many other benefits.

Accordingly one treatment method is to deliver a functional volume of the formulation of the present invention intravenously to enable particular photonic wavelengths of photonic energy to be absorbed, reformed and then delivered by the C60, which is re-released in the body in the red spectrum to further enhance the therapeutic properties of the formulation of the present invention.

### EXAMPLES

Various products are produced by the processes and methods disclosed herein.

### Example #1

A C60 colloid and cannabinoid formulation including 25 mg CBD 5 mg C60 in 2 ml coconut oil packaged in a orally consumable capsule.

### Example #2

A C60 and cannabinoid colloid including 25 mg CBD 5 mg C60 in 2 ml coconut oil packaged in a suppository capsule.

### Example #3

A colloid including 50 mg CBD 10 mg C60 in 2 ml coconut oil emulsion for topical application.

### Example #4

A colloid including 10 mg THC and 2 mg C60 in an aqueous solution for nasal spray delivery.

### Example #5

A suspension including 10 mg THC and 10 mg CBD in the form of a whole plant extract cannabis oil and no carrier oil, including 5 mg C60.

### Example #6

A formulation including a lipid excipient, whole plant extract cannabis flower oil, and C60 where the ratio of active cannabinoids to C60 is 2:1.

### Example #7

A colloid including a lipid excipient, whole plant extract cannabis flower oil, and C60 where the ratio of active cannabinoids to C60 is 2:1, and wherein the lipid excipient is coconut oil.

### Example #8

A formulation including a lipid excipient, whole plant extract cannabis flower oil, and C60 where the ratio of active cannabinoids to C60 is 4:1, and wherein the lipid excipient is coconut oil.

### Example #9

A formulation including an engineered lipid excipient having medium chain lipids, whole plant extract cannabis flower oil, and C60 where the ratio of active cannabinoids to C60 is 4:1, and wherein the lipid excipient is coconut oil, and the active cannabinoids are predominately CBD.

### EXAMPLE #10

coconut oil excipient solution including CBD and C60 in a ratio within the range of 1:10 to 10:1 on a weight to weight w/w basis. The formulation has a at least one mg of CBD per ml of the formulation.

All ratios are expressed in this document are on a w/w basis, unless otherwise noted.

We claim:

1. A cannabinoid formulation having improved shelf life comprising:
cannabidiol in a concentration of at least one milligram per ml;
an oil-based excipient; and
a molecule having the chemical formula C60.

2. The formulation as set forth in claim 1, wherein the ratio of the cannabidiol to the C60 is between 10:1 and 1:1.

3. The formulation as set forth in claim 2, wherein the excipient is solely coconut oil.

4. The formulation as set forth in claim 2, wherein the excipient includes coconut oil.

5. The formulation as set forth in claim 2, wherein the cannabidiol is isolated Cannabidiol.

6. The formulation as set forth in claim 2, wherein the cannabidiol is predominately cannabidiolic acid.

7. A cannabinoid formulation comprising:
tetrahydrocannabinol in a concentration of at least one milligram per ml of the cannabinoid formulation;
an oil-based excipient; and
a molecule having the chemical formula C60.

8. The formulation as set forth in claim 7, wherein the ratio of the tetrahydrocannabinol to the C60 is between 10:1 and 1:1.

9. The formulation as set forth in claim 8, wherein the excipient includes coconut oil.

10. The formulation as set forth in claim 8, wherein the excipient is only coconut oil.

11. The formulation as set forth in claim 8, wherein the tetrahydrocannabinol is isolated tetrahydrocannabinol.

12. The formulation as set forth in claim 8, wherein the tetrahydrocannabinol is predominately terahydrocannabinoic acid.

13. The formulation as set forth in claim 8, wherein the C60 is hydrogenated.
